(19) United States
(12) Patent Application Publication
(10) Pub. No.: US 2007/0073218 A1
(43) Pub. Date: Mar. 29, 2007

(54) Inflatable cardiac device for treating and preventing ventricular remodeling

(76) Inventors: Lilip Lau, Los Altos, CA (US); Craig Mar, Fremont, CA (US); Anh Truong, Fremont, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/235,684

(22) Filed: Sep. 26, 2005

(51) Int. Cl. A61M 31/00

(52) U.S. Cl. 604/93.01

(57) ABSTRACT

An inflatable cardiac device for placement in the pericardial sac is described. The inflatable cardiac device is useful for treatment of congestive heart failure and for attenuating or reversing remodeling of the heart. In some embodiments, the device further includes filling catheters connected with reservoirs, on-off devices, or pressure transducers. The device may also include a drug delivery system. At least one embodiment includes electrodes capable of connection with an implantable electronic medical device such as a pacemaker or defibrillator.

### FIELD OF THE INVENTION

The present invention relates to mechanical systems for treating congestive heart failure. Specifically, the invention relates to devices that interface mechanically with a patient's failing heart in order to improve intrinsic cardiac function.

### BACKGROUND OF THE INVENTION

Congestive heart failure (“CHF”) is characterized by the failure of the heart to pump blood at sufficient flow rates and pressures to meet the metabolic demand of tissues, especially the demand for oxygen. Historically, congestive heart failure has been managed with a variety of drugs. There is also a considerable history of the use of devices to improve cardiac output. For example, physicians have employed many designs for powered left-ventricular assist pumps. Multi-chamber pacing has been employed to optimally synchronize the beating of the heart chambers to improve cardiac output.

Various skeletal muscles have been investigated as potential autologous power sources for ventricular assist. Among these, dynamic cardiomyoplasty using the latissimus dorsi muscle has attracted the most interest. It has been suggested that the beneficial effects of this procedure stem from both an active, dynamic, systolic assistance and a passive, adynamic girdling of the heart that limits diastolic stretch of the ventricle.

To exploit these beneficial clinical features, researchers and cardiac surgeons have experimented with prosthetic “girdles” around the heart. One such design reported in the literature is a prosthetic “sock” that is wrapped around the heart. Others have proposed the application of an intraventricular splint to reduce the volume of the left ventricle. Several design shortcomings are apparent with each.

The intraventricular splint, for example, extends through the left ventricular wall. Consequently, some components of the splint contact the patient's blood. This creates the potential for thrombogenesis, or the generation of blood clots. The blood clots may thromboembolize and clog peripheral arteries with potential of stroke among other serious vascular problems. In addition, splint placement requires perforation of the ventricular wall, which may lead to leakage problems such as hemorrhage or hematoma formation. Furthermore, because one end of the splint extends to the epicardial surface of the left ventricle, options for the orientation of the splint are limited.

Pulling opposite walls of the ventricle closer together may reduce average wall stress via LaPlace's law, by reduction in ventricular diameter. However, this may create an irregular ventricular wall contour. This creates stress concentrations in the regions of the ventricle that are between the localized compression points. Consequently, this may lead to aneurysm formation, fibrosis, and impairment of the contractility and compliance of the ventricle. Also, the resulting irregular contour of the endocardial surface of the left ventricle may lead to localized hemostasis or turbulence, which may in turn lead to thrombus formation and possible thromboembolism.

Coronary artery disease causes approximately 70% of congestive heart failure. Acute myocardial infarction (“AMI”) due to obstruction of a coronary artery is a common initiating event that can lead ultimately to heart failure. This process by which this occurs is referred to as remodeling and is described in the text Heart Disease, 5th ed., E. Braunwald, Ch. 37 (1997). Remodeling after a myocardial infarction involves two distinct types of physical changes to the size, shape and thickness of the left ventricle. The first, known as infarct expansion, involves a localized thinning and stretching of the myocardium in the infarct zone. This myocardium can go through progressive phases of functional impairment, depending on the severity of the infarction. These phases reflect the underlying myocardial wall motion abnormality and include an initial dyssynchrony, followed by hypokinesis, akinesis, and finally, in cases that result in left ventricular aneurysm, dyskinesis. This dyskinesis has been described as “paradoxical” motion because the infarct zone bulges outward during systole while the rest of the left ventricle contracts inward. Consequently, end-systolic volume in dyskinetic hearts increases relative to nondyskinetic hearts.

The second physical characteristic of a remodeling left ventricle is the attempted compensation of noninfarcted region of myocardium for the infarcted region by becoming hyperkinetic and expanding acutely, causing the left ventricle to assume a more spherical shape. This helps to preserve stroke volume after an infarction. These changes increase wall stress in the myocardium of the left ventricle. It is thought that wall tension is one of the most important parameters that stimulate left ventricular remodeling (Pfeffer et al. 1990). In response to increased wall tension or stress, further ventricular dilatation ensues. Thus, a vicious cycle can result, in which dilatation leads to further dilatation and greater functional impairment. On a cellular level, unfavorable adaptations occur as well. This further compounds the functional deterioration.

Some have proposed that an elastic wrap around the heart might attenuate the remodeling process that is actively underway in failing hearts, prompting treatment with latissimus dorsi cardiomyoplasty. Based on experimental work to date, passive latissimus dorsi muscles appear to be best suited for this application. Oh et al. (1997) published experimental work in which they found a relatively inelastic prosthetic fabric wrap to be inferior to adynamic latissimus dorsi in bringing about reverse remodeling in an experimental model of heart failure. This was attributed to the greater elasticity of the muscle wrap.

It is thought that application of a device to provide compressive reinforcement similar to that of adynamic cardiomyoplasty might be therapeutic in treating dilated, failing hearts. Because heart failure is only the clinical end-stage of a continuous remodeling process, such a device might be able to attenuate or stop remodeling after a myocardial infarction far before the onset of heart failure. Such a device would have different functional requirements from a device that is used solely to treat established heart failure. Furthermore, such a device is beneficial for cardiac function without needing to provide an auxiliary dynamic pumping mechanism.

One requirement is to provide a slight elastic compression to the epicardial surface of the left ventricular wall. The device should allow expansion and contraction of the heart, but continue to apply gentle elastic compression to the left ventricle. This would reduce circumferential and longitudinal wall tension, thereby improving efficiency, lowering energy expenditure, reducing neuro-hormonal activation, encouraging favorable cellular changes, and stabilizing the dimensions of the heart. This mechanical action is often referred to as “myocardial sparing.”

Preferably, the device should effect myocardial sparing without limiting the motion or the dimensions of the heart. Nor should it actively change the shape of the heart by pulling it or squeezing it. In fact, imposing a rigid barrier to limit distension or to squeeze the heart can be potentially dangerous. Shabetai in The Role of the Pericardium in the Pathophysiology of Heart Failure notes that the pericardium exerts 3-4 mm Hg of pressure against the heart. Cardiac function can be adversely affected with just a slight increase in pericardial constraint. For example, cardiac tamponade begins to be seen with pericardial pressures as low as 5-10 mm Hg.

A second requirement of such a device is to provide reinforcement that prevents the further shape change of the left ventricle without acutely changing the shape by its application. The device would act to prevent both global dilatation toward a more spherical shape and local infarct expansion after a myocardial infarction. In fact, if only the local infarct expansion can be minimized with such a device, the compensatory global dilatation and increase in sphericity may be prevented. Therefore, a circumferential compression of the myocardium may not be necessary to prevent the increase in sphericity.

What is needed is a mild compressive support the contour of which conforms to the damaged portion of the heart. As the left ventricle or portions of the left ventricle distend outward, they would be met with greater pressure from the device. The presence of the device would likely cause the left ventricle to reverse-remodel and its dimensions to stabilize and even shrink. As this occurs, the device, preferably, would be capable to follow the contour of the left ventricle, as seen with latissimus dorsi muscle. The device would also preferably supply less pressure on the myocardium as the diameter of the heart decreases. Conversely, the device would supply gradually increasing pressure as the diameter of the heart or local distention increases. This ideal was expressed by Oh et al. in their description of the benefits of a passive latissimus dorsi muscle wrap.

The ability of the device to conform to the heart as it shrinks or expands is of great importance. A circumferential device would need to possess considerable elasticity in order to do so. However, if an inflatable device were implanted between the pericardium and the epicardium, the pericardium would naturally apply the device against the epicardial contour. Furthermore, an inflatable device could have volume added or removed to the device to adjust device size, shape, and pressure. An inflated device would provide mild compressive support and conform to the contour of the damaged portion of the heart. An inflated device would also naturally apply gradually increasing counter-pressure against the heart, as the heart expanded and pressed against the inflated device.

The left ventricle in a dilated, failing heart does not distend significantly because small diameter changes are sufficient to achieve the necessary stroke volume. In contrast, a normal heart has a much smaller left ventricular diameter. For example, Li (1997) noted that to achieve a 70-cc stroke volume, a normal left ventricle of 2.8 cm radius contracts down to 1.7 cm, a 40% decrease. However, a dilated ventricle of 4.5-cm radius achieves the same stroke volume by contracting to 4.2 cm, only a 7% decrease. Thus, in order to achieve the same stroke volume as a dilated heart, the normal heart's ventricular diameter must change by a greater amount. Consequently, a device with sufficient elasticity for treating dilated hearts in established heart failure may not be able to treat a heart of normal dimensions that has suffered a myocardial infarction.

The ability of an inflatable pericardial device to conform to the heart is also theoretically important in preventing dilated heart failure after acute myocardial infarctions because it may be important to provide reinforcement during systole, especially early systole. In addition to providing more myocardial sparing over a greater portion of the cardiac cycle, a device that remains in compressive contact with the heart into systole would counteract the “paradoxical bulging” of the infarct region that occurs in dyskinetic, aneurysmal hearts during systole. This may attenuate infarct expansion and therefore limit the extent of remodeling that further ensues.

Since the mid 1980's a promising procedure has been evaluated clinically. The procedure, dynamic cardiomyoplasty, involves surgically dissecting the patient's latissimus dorsi muscle, introducing it into the thoracic cavity, and then wrapping and attaching the muscle to the heart. An implantable electrical stimulator is connected to the muscle in order to stimulate and pace it in synchrony with the heart. This causes the muscle to contract and also transforms the muscle, making it more fatigue-resistant. The original premise behind dynamic cardiomyoplasty was that these muscle contractions, by virtue of the geometry of the wrap, would squeeze the heart, and thus provide systolic assistance. If successful, an essentially patient-powered, relatively inexpensive, non-blood-contacting, easily placed ventricular-assist device could be employed.

The first reported clinical case of dynamic cardiomyoplasty using a latissimus dorsi wrap was published in 1985. Since then, over 1,000 patients have been treated with this experimental procedure. Numerous published studies have shown that the procedure produces significant improvement in clinical status, as graded by the New York Heart Association (“NYHA”) classification scale, a slight but significant hemodynamic or systolic function improvement, and a reduction in the number of patient hospital visits after the procedure. However, an improvement in survival has yet to be consistently demonstrated. Furthermore, perhaps due to their frail condition, NYHA class IV patients have not fared well with the procedure. This has limited its use to NYHA class III patients. It appears that the skeletal muscle wrap, probably because of its deterioration over time, does not provide sustained squeezing of the heart over time. Yet, the clinical benefits of the procedure appear to persist. This paradox has led to considerable research into the underlying mechanisms of dynamic latissimus dorsi cardiomyoplasty.

This research has resulted in several independently additive hypothetical mechanisms to explain the benefits of dynamic cardiomyoplasty. The original concept of systolic squeezing of the heart, in particular the left ventricle, was shown in experimental work to provide hemodynamic benefit. But there additionally appears to be a considerable benefit derived from the presence of the passive, unstimulated latissimus dorsi wrap alone. Drs. Chiu (1992), Carpentier (1993), and others hypothesized that the presence of the latissimus dorsi wrap provides a beneficial passive function beyond, the benefits of systolic-squeezing augmentation. It was speculated that the muscle wrap acts as a girdle around the heart. The girdle is thought to impose a physical limit on the heart to prevent it from dilating beyond its boundaries. This is commonly referred to as the “girdling” effect.

A separate and equally powerful hypothesis was that the muscle wrap helps the native myocardium bear some of the load, in essence reducing myocardial tension or wall stress, via Laplace's law, by creating a thicker wall. This has been referred to as the “myocardial sparing” effect by virtue of the reduction in wall stress and concomitant reduction in oxygen consumption. The benefits of these two passive mechanisms are thought to be additive with the systolic squeezing benefits of cardiomyoplasty. Published experimental work by Nakajima et al. (1994), Chen et al. (1995), Kawaguchi et al. (1992 & 1994), Kass et al. (1995), Capouya et al. (1993), Chekanov (1994) and others provide support to the validity of the hypothetical mechanisms.

Experimentally, passive, unstimulated latissimus dorsi cardiomyoplasty wraps appear to be the best at attenuating remodeling and heart failure. However, in a clinical setting, the surgery required to dissect and attach the muscle around the heart is very extensive and traumatic. Even if such a therapy were proven clinically efficacious, this factor limits its potential acceptance.

A cardiac harness for supporting and gently compressing the heart, thereby attenuating remodeling of the myocardium, is described in U.S. Pat. No. 6,595,912 to Lau et al. The harness described in that patent includes a plurality of interconnected elastic bending hinges, each of which has a central portion connected on opposite sides to respective arm portions. The arm portions interact with the central portion in response to deflection of the arm portions to create a bending moment in the hinge to store potential energy. The harness may be inserted by a minimally invasive technique, thus avoiding sternotomy.

The cardiac harness also has certain disadvantages. Once the cardiac harness is in place, the pressure that the harness exerts upon the myocardium cannot be adjusted. The surgeon must therefore either carefully predetermine the size and shape of the harness, or be prepared to make multiple passes until a properly fitting cardiac harness is finally in place. Furthermore, the cardiac harnesses in the prior art must be placed circumferentially around the heart in order to be effective. This is a significant disadvantage, requiring manipulation of anterior, posterior, and lateral surfaces of the heart. Extensive dissection of the pericardium is necessary in order to slip the cardiac harness onto the heart.

Inflatable devices to assist the heart have previously been described in as auxiliary pumping mechanisms to assist contraction of the myocardium. Inflatable cardiac devices have therefore been active, rather than a passive devices for the heart. An example of an active inflatable pumping device is U.S. Pat. No. 6,699,259 to Fogarty. Forgarty describes an inflatable device useful for Cardiopulmonary resuscitation (CPR). The device is inserted to temporarily massage the heart over the short term, but is not designed to be implantable. After insertion into the sternocostal space and removal of an insertion sleeve, the Fogarty bladder is repeatedly inflated and deflated to massage the heart and provide blood flow.

Fluid filled inflatable cardiac devices are usually connected with an external motor driven pump. These types of devices alternatively compress and relax the myocardium, thus providing cyclic cardiac compression. These fluid filled external cardiac pumps therefore mostly surround the heart and work by exerting dynamic circumferential pressure against the myocardium. Alternatively, some of the other dynamic pumping devices include opposing plates with attached inner facing expandable chambers which alternately compress and release the myocardium between the plates utilizing an external pumping mechanism.

U.S. Pat. No. 6,432,039 to Wardle discloses a method and apparatus for reinforcement of the heart ventricles. The device includes a circumferential frame or containment structure and inflation pockets with recoil balloons. The frame or containment structure is not elastic and cannot be delivered with minimally invasive surgery. The containment structure includes slits that must be laced up using an open surgical procedure. Furthermore, the inflation pockets and recoil balloons are in a fixed configuration within the containment structure, therefore limiting the clinicians flexibility in the positioning of the device against chosen areas of the heart.

Cardiac harnesses or jackets described in the known prior art are circumferential devices. Cardiac harness or jackets therefore have a potential disadvantage of providing a circumferential tension or pressure around the entire heart and are not capable of applying tension or pressure to selected areas of the heart. Another disadvantage of harnesses or jackets is that they cover the coronary arteries, making surgical access to the coronary arteries difficult of impossible at a later date.

### SUMMARY OF THE INVENTION

Accordingly, a need exists for an implantable cardiac device for reversing and attenuating cardiac remodeling that overcomes the disadvantages of the prior art. One aspect of the present invention includes an inflatable cardiac device for treating or preventing congestive heart failure and deleterious cardiac remodeling associated with myocardial infarction. The inflatable device chamber is preferably placed between the pericardium and the epicardium and can be delivered with standard open thoracotomy, or preferably with minimally invasive surgical technique. For example, the invention provides a device that can be rolled up and delivered through a canula for implantation within a patient. The inflatable device may also be delivered percutaneously, for example, through a subxiphoid approach. In at least one embodiment, the device is positioned upon the heart using non-invasive imaging, for example echocardiography and/or fluoroscopy. The device may be delivered using echocardiography to visualize and guide placement of the device. In one embodiment the device is at least partially radiopaque. The device may include radiopaque radiographic markers. A partially radiopaque device may be placed using fluoroscopy to visualize and guide placement of the inflatable device. Advantageously, the inflatable device does not need to be placed circumferentially around the heart. The inflatable device may be placed selectively over a portion of the heart, for example, the portion affected by cardiac infarction. The inflatable device may also be placed in a manner that avoids covering over the coronary arteries. More than one inflatable cardiac device may be placed in a patient.

In at least one embodiment, the inflatable chamber has elastic qualities. In other embodiments, the inflatable chamber may be relatively stiff. The inflatable chamber may be filled with a gas, a liquid, a gel, or sponges. In some embodiments, flow of filling substance between the inflatable chamber and a reservoir will provide pressure regulation and elastic qualities to the invention.

The present invention provides a fluid filled prosthetic device that reduces wall stress by maintaining compressive contact against the epicardium over a significant portion of the cardiac cycle. The device is safely implantable over extended periods of time. Preferably, the device does not need to be removed unless there is a complication. The inflatable device may include an exterior surface having an increased coefficient of friction, whereby the inflatable chamber resists sliding over the epicardial surface of the heart. The invention in at least one embodiment provides anchoring members on the exterior of an inflatable chamber. The invention does not require an additional support, frame, or shell to stay in place against the heart.

In at least one embodiment, the invention provides a fluid filled prosthetic device wherein pressures exerted against the myocardium may be easily adjusted during or after implantation. The clinician can inject or withdrawn fluid from within the device during implantation of the device or at a later time. Changes in fluid volume within the device can allow for a corresponding change in the pressures exerted against the myocardium. An injection port or a reservoir are provided in some embodiments so that filling substance may be conveniently added or removed from the device without requiring a subsequent invasive procedure.

In at least one embodiment, the invention provides a prosthetic device that reduces focal wall stresses by maintaining compressive contact against a designated part of the epicardium. Furthermore, the device advantageously is capable of applying pressure against the heart without the need to apply a circumferential harness. Circumferential dissection around the heart is not needed to place the inflatable cardiac device. The inflatable device can be made in various sizes and shapes. In some embodiments, multiple compartments allow flexibility in applying compression to only a limited portion of the heart. In at least one embodiment, multiple channels may extend between the epicardial and the pericardial surfaces of the inflatable device, thereby allowing tissue growth through the channels. The channels may also be beneficial in permitting defibrillation of the heart by providing a discontinuous covering over the surface of the heart.

The invention provides a device that is compact enough to be delivered through a very small and minimally invasive incision, and then inflated to the desired size after placement. An inflatable chamber has a first compressed configuration for minimally invasive delivery and a second expanded configuration, following placement in the pericardial sac. For example, the inflatable chamber can be rolled up and delivered into the pericardial sac through a canula. In yet another embodiment, the inflatable chamber may be delivered in other collapsed configurations, for example, folded up upon itself. In one embodiment, the inflatable chamber may also be delivered percutaneously via an infracardiac subxiphoid approach. The inflatable chamber can be inflated and expanded to a desired size and pressure after it is placed in a final location in the pericardial sac.

In certain preferred embodiments, the interior of the inflatable device chamber may be filled with various quantities and types of liquids, gases, or gels. The interior of the inflatable device may also be filled with a sponge. The volume occupied by the device and pressures delivered by the device will vary depending on the amount and type of the filling substance used to fill the chamber, the elasticity of the chamber walls, and the compliance of the inflatable device. In at least one embodiment, the device is filled with normal saline. In other embodiments, the device is filled with a gel, for example, a silicone gel. In other embodiments, the device is filled with a gas for increased elasticity. In yet other embodiments, the device is filled with a polymer or a sponge. The device may come pre-filled from the manufacturer, or could be filled by the surgeon, at the time of implantation. In some embodiments, it is advantageous to include filling catheters to allow the clinician to fill or remove filling substance via subcutaneous access, or exchange one type of filling substance for another.

In some embodiments, one or more electrodes may be attached to an exterior surface of the inflatable chamber, such that when the inflatable device is implanted, the electrodes will make electrical contact the heart. Wire leads connected with the electrodes may then be connected to an implantable device for pacing, for cardioversion, of for measuring the impedance across the heart.

In some preferred embodiments, the device may further include one or more filling catheters. The filling catheter includes a distal end, which attaches to and is in fluid communication with the interior of an inflatable chamber. The proximal end of the catheter, which is the end farthest from the chamber, may be attached for example to a syringe in order to fill the chamber with fluid. The catheters, preferably, are of sufficient length to allow each to extend at least from the chamber to the surgical skin incision site.

In another aspect of the invention, the addition or withdrawal of volume should preferably be capable of being performed with minimally invasive or non-invasive techniques. In at least one embodiment, subcutaneous reservoirs are connected with the filling catheters. The reservoirs could be filled, or fluid removed from the reservoir, and thereby the inflatable chambers, by percutaneous needle puncture of the reservoir.

In yet another embodiment, a unidirectional valve may be placed between the inflatable chamber and the reservoir. The addition of a valve permits fluid to flow in only one direction within a catheter. An advantage of a valve, in some embodiments, is that fluid in the reservoir may be forced into or removed from the inflatable chamber, utilizing only fluid already in the system, and thus avoiding the need for needle puncture of the reservoir. In other embodiments, valves that allow flow only at predetermined pressures can be used to regulate pressure within the inflatable chamber.

In still other embodiments, a drug delivery catheter may be included with the device for delivery of drugs external to the inflatable device. The drugs would be deliverable directly to the pericardial space. The catheters may also communicate with a reservoir or an active drug pump that delivers the drug in a periodic bolus or by a continuous infusion method. The catheter could also deliver an adhesive gel to the exterior of the inflatable chamber to secure the chamber in place next to the heart.

In at least one embodiment, the invention will include a pressure transducer and a transmitter. The clinician may be able to monitor the pressure within the chamber by for example wireless communication techniques known in the art. This will enable the clinician, with knowledge of the pressure within the chamber, to add or remove fluid in order to customize the pressure delivered to the patient's heart.

Another aspect of the invention is a method of treating a diseased heart with minimally invasive surgery. The method of treating a diseased heart, comprises providing an inflatable cardiac device having at least one inflatable compartment and inserting the inflatable cardiac device into a patient. The method further includes positioning the inflatable cardiac device in a collapsed configuration between the patient's epicardium and pericardium and inflating the inflatable chamber with a filling substance. The method further includes leaving the inflatable cardiac device implanted within the patient. The inflatable cardiac device is configured to be implanted within the patient for weeks, months, or even years.

Further features and advantages of the present invention will become apparent to one of skill in the art in view of the Detailed Description of the Preferred Embodiments which follows, when considered together with the attached drawings and claims

### BRIEF DESCRIPTION OF THE DRAWINGS

These and other features, aspects and advantages of the present invention are described with reference to drawings of preferred embodiments, which are intended to illustrate, but not to limit, the present invention.

FIG. 1A is an illustration of an inflatable chamber in location against a heart within a pericardial sac.

FIG. 1B shows an inflatable chamber in a deflated collapsed configuration.

FIG. 1C shows an inflatable chamber in an inflated expanded configuration.

FIG. 1D is a cross sectional view illustrating an inflatable chamber in a deflated collapsed configuration with the inflatable chamber rolled up inside a canula.

FIG. 1E is a perspective view of a portion of the inflatable chamber and canula of FIG. 1D.

FIG. 2A is a cross sectional view of an inflatable chamber expanded against a heart of a patient.

FIG. 2B is a cross sectional view of an inflatable chamber expanded against a heart of a patient.

FIG. 2C is a cross sectional view of an inflatable chamber having a supplemental layer, the inflatable chamber having been expanded against a heart of a patient.

FIG. 3A is a perspective view illustrating an inflatable chamber having anchoring members on an exterior surface.

FIG. 3B is a cross sectional view of two inflatable chambers expanded against a heart, one of the inflatable chambers having anchoring members on a medial chamber wall and the other inflatable chamber having anchoring members on both the medial chamber wall and a lateral chamber wall.

FIG. 4 is a perspective view of an inflatable cardiac device including a filling catheter.

FIG. 5 is a perspective view of an inflatable cardiac device illustrating the proximal end of a filling catheter connected with another member selected from an injection port, a reservoir, an on-off device, a one way valve, and a pressure transducer.

FIG. 6A is perspective view illustrating an inflatable cardiac device including two inflatable compartments of generally equal size, both compartments having a narrow width.

FIG. 6B is perspective view illustrating an inflatable cardiac device including two inflatable compartments of generally unequal size, one compartment having a narrow width and one compartment having a wide width.

FIG. 6C is perspective view illustrating an inflatable cardiac device including two inflatable compartments of generally equal size, both compartments having a wide width.

FIG. 6D is sectional perspective view of a portion of an inflatable cardiac device wherein multiple inflatable compartments are configured as concentric rings.

FIG. 7A is a cross sectional view of an inflatable chamber including interconnecting members between a medial chamber wall and a lateral chamber, wherein the inflatable chamber is not expanded.

FIG. 7B is a cross sectional view of the inflatable chamber of FIG. 7A, wherein the chamber has been further expanded.

FIG. 7C is a cross sectional view of the inflatable chamber of FIG. 7A, wherein the chamber has been fully expanded.

FIG. 8A is a plan view of a portion of an inflatable cardiac device including a drug delivery catheter.

FIG. 8B is a plan view of a portion of an inflatable cardiac device including a drug delivery catheter and further including a baffle system for distribution of a drug along a surface of an inflatable chamber.

FIG. 9 is a perspective view showing an inflatable cardiac device mounted on a heart, the inflatable cardiac device further including an electrode (not shown) having at least one lead connected to an implantable electronic medical device, and an external receiver/transmitter in communication with the implantable electronic medical device.

FIG. 10 is a plan view of an inflatable chamber having a plurality of narrow compartments.

FIG. 11 is a plan view of an inflatable chamber having a plurality of wide compartments.

FIG. 12 is a plan view of an inflatable cardiac device having a sensing electrode and two defibrillating electrodes.

FIG. 13 is a cross sectional view taken through line 13 of FIG. 12.

FIG. 14 is a perspective view of the inflatable cardiac device of FIG. 12 in a collapsed rolled up configuration.

FIG. 15 is a plan view of an inflatable chamber having channels traversing through a lateral chamber wall and a medial chamber wall.

FIG. 16 is a cross sectional view through line 16 of FIG. 15.

FIG. 17 is a perspective view of an inflatable cardiac device including a pouch configured for the insertion of multiple inflatable compartments.

FIG. 18 is a plan view of an inflatable cardiac device including sheaths configured for the insertion of delivery rods.

FIG. 19 is a plan view of the inflatable cardiac device of FIG. 18 showing three delivery rods inserted into the sheaths.

FIG. 20 is a cross sectional view through a portion of the inflatable cardiac device of FIG. 18 showing the delivery rod inserted into the sheath.

### DETAILED DESCRIPTION OF THE PREFERRED EMBODIMENTS

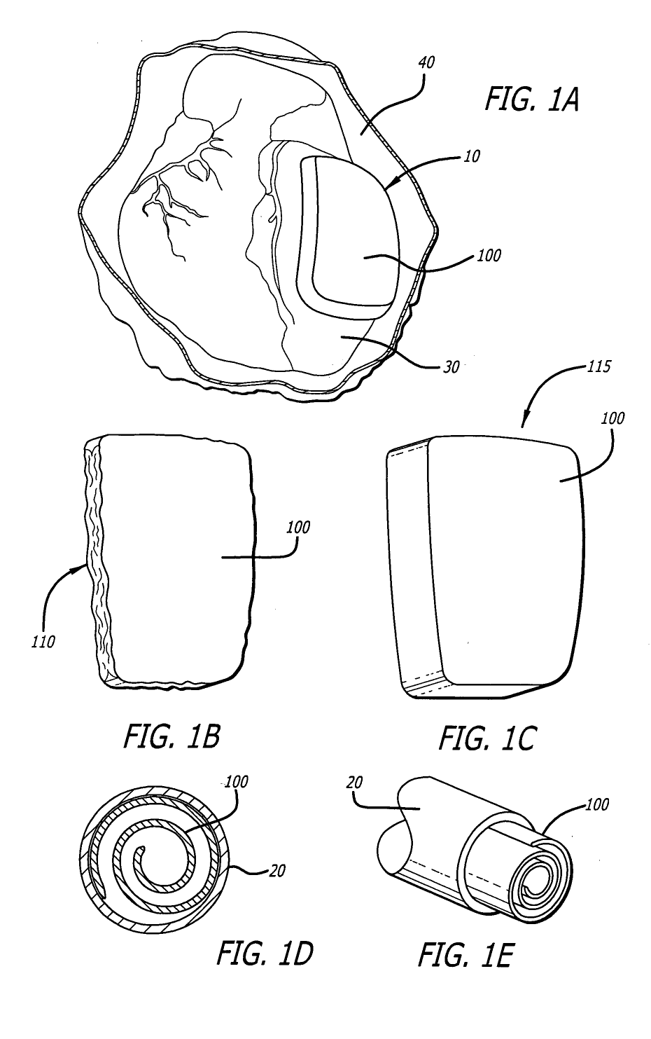

Referring first to FIG. 1A, the present invention is an inflatable cardiac device 10 including an inflatable chamber 100 for implantation adjacent to the heart. The inflatable cardiac device 10 is designed for treating remodeling of the heart and preventing or reversing deleterious effects on cardiac function associated with congestive heart failure. The inflatable cardiac device is configured to attenuate and/or reverse remodeling. The inflatable cardiac device in at least one embodiment is implanted between the epicardium 30 and the pericardium 40, within the pericardial sac. As illustrated in FIG. 1B, the inflatable chamber 100 has a first collapsed or deflated configuration 110 for minimally invasive delivery and as illustrated in FIG. 1C, a second expanded configuration 115. The inflatable chamber 100 is preferably expanded following placement in the pericardial sac, although it may also be expanded before placement in the pericardial sac. In at least one embodiment, the inflatable chamber 100 is also deflatable and is configured to be collapsible after inflation. The inflatable chamber 100 is preferably made of a soft medical grade implantable material, for example silicone. The inflatable chamber 100 may also be made of polyurethane or expanded polytetrafluoroethylene (ePTFE)(Gortex™). The inflatable cardiac device may also be made from other biocompatible materials well known in the art. The inflatable cardiac device is capable of long term implantation in the patient because the inflatable cardiac device is configured to remain in a patient for weeks, months, or even years.

Referring now to FIG. 1D and FIG. 1E, the deflated inflatable chamber 100 may, for example, be rolled up and placed in a canula 20 for minimally invasive delivery into the pericardial sac. In yet another embodiment, the inflatable chamber 100 may be delivered in other collapsed configurations, for example, folded up upon itself. In one embodiment, the inflatable chamber 100 may also be delivered percutaneously via an infracardiac subxiphoid approach. After the deflated inflatable chamber 100 is adjacent to the heart, the inflatable chamber 100 is then expanded by filling the inside of the inflatable chamber 100 with a filling substance. The filling substance is preferably fluid and may be a gas, a liquid, a gel. In one embodiment, the filling substance does not need to be fluid and may also be other compressible materials, for example a sponge. The inflatable chamber 100 is capable of retaining the filling substance within its walls. At least one embodiment is intended to be implanted and retained between the pericardium and the epicardium. In an expanded configuration, the inflatable chamber 100 exerts a gentle elastic pressure upon the myocardium. Once the distension of the pericardium has reached a critical level by lateral pressure of the inflatable chamber against the pericardium, the inflatable chamber 100 will begin to exert compressive forces against the myocardium.

In at least one embodiment, the device is positioned upon the heart using non-invasive imaging, for example echocardiography and/or fluoroscopy. In one embodiment, the inflatable chamber 100 is echogenic. The inflatable chamber may be placed using echocardiography for guidance. In one embodiment, the inflatable chamber 100 is at least partially radiopaque. The inflatable chamber 100 may include radiopaque radiographic markers (not shown). The inflatable chamber may therefore be placed with the assistance of fluoroscopy because it is at least partially radiopaque.

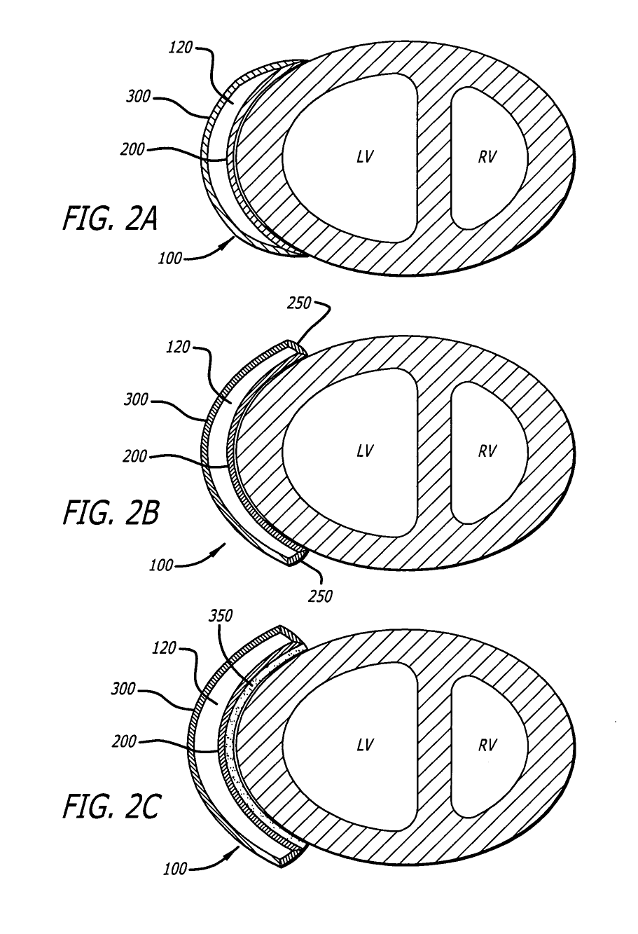

Referring now to FIG. 2A, in one embodiment, the implantable inflatable chamber 100 includes at least two opposing chamber walls, said walls enclosing a cavity 120 that can be inflated with the filling substance. The chamber walls in at least one embodiment include a medial chamber wall 200 and a lateral chamber wall 300. The medial chamber wall 200 is the chamber wall of the inflatable cardiac device 10 that is intended to be closest to the epicardium after implantation. The lateral chamber wall 300 is closest to the pericardium after implantation of the inflatable chamber 100. As shown in FIG. 2A, in some embodiments, the periphery of the lateral chamber wall 300 may be directly connected to the periphery of the medial chamber wall 200. Connections between wall peripheries may be achieved by heat seal or application of an adhesive. Other methods of bonding well known in the art are also applicable. As shown in FIG. 2B, in yet other embodiments, one or more peripheral walls 250 may further be included. The peripheral walls 250 may connect an edge of the medial chamber wall 200 to an edge of the lateral chamber wall 300. The peripheral walls 250 are advantageous in providing a thicker width and rounder gentle contour to the inflatable chamber 100.

The medial chamber wall 200 and the lateral chamber wall 300 are preferably made from soft implantable medical grade materials, for example silicone or plastics. The chamber walls may also be made of polyurethane or expanded polytetrafluoroethylene (ePTFE)(Gortex™). The chosen materials are those known in the art to be appropriate for long term implantation in the human body. In one embodiment, the inflatable chamber 100 is configured to remain in the body long term, for example weeks, months, or years, unless a medical complication requires the inflatable chamber 100 to be removed. In one embodiment, the inflatable chamber 100 may be deflated and removed from the patient at a later date. Furthermore, the inflatable chamber 100 may be deflated and left inside the patient in the deflated configuration. However, of course, the inflatable chamber 100 is also capable of remaining inside the patient for only a shorter time.

The medial chamber wall 200 and lateral chamber wall 300 may be made of the same type, size, and thickness of material in some embodiments. In other embodiments, the medial chamber wall 200 and the lateral chamber wall 300 may differ as to chemical composition or thickness. For example, the medial chamber wall 200 can be made stiffer than the lateral chamber wall 300, or vice versa. By varying the expansion properties of the medial chamber wall 200 and the lateral chamber wall 300, in these various embodiments, the contour of the implantable chamber 100 and the dynamics of myocardial compression can be varied. Furthermore, at least one of the chamber walls may be textured either to alter the dynamic expansion properties of the inflatable chamber 100 or to increase the coefficient of friction to promote gripping or adhesion of the inflatable chamber 100 to the epicardial and/or pericardial surfaces.

Referring now to FIG. 2C, in some embodiments, the inflatable cardiac device 10 may further include a supplemental layer 350 which can be connected to the medial chamber wall 200 or the lateral chamber wall 300. The supplemental layer 350 may be attached using, for example, an adhesive or heat bonded into place. The supplemental layer 350 in some embodiments may include various medical grade materials that add stiffness or reinforce the cardiac wall. The supplemental layer 350, in yet other embodiments, may include biologic materials, for example homographs, allographs, cell cultures, sheets of collagen, or muscle. Additional supplemental materials may be held in place by compression of the inflatable chamber 100 against the epicardium 30. Alternatively, in other embodiments, the supplemental layer 350 may include a patch containing a pharmacologically active substance or drug. Drugs may advantageously then be delivered by direct application to the epicardium and myocardial wall.

Preferably, the inflatable chambers are not positioned directly over the coronary arteries, thereby improving later surgical access to the coronary arteries if required, for example, for bypass surgery. It is advantageous in clinical use to secure the inflatable chamber 100 in a fixed position after implantation in a patient. If the inflatable chamber 100 were to slide out of position, the inflatable chamber might become ineffective or unintentionally compress other organs, vessels, or undesirable parts of the heart. In some patients, this might be achieved by careful dissection of the pericardium during implantation, and thereafter carefully wedging the inflatable chamber 100 between the epicardium 30 and pericardium 40. However, the invention includes in some embodiments other ways for securing the inflatable device 10 in place. The exterior surface of the inflatable chamber is preferably configured with an increase coefficient of friction. In at least one embodiment, as described above, a textured surface on the exterior of the inflatable chamber 100 helps increase the coefficient of friction and thereby secure the inflatable chamber 100 in the pericardial sac. The inflatable chamber 100 may be further secured in place with biocompatible adhesives. In yet other embodiments, other methods of increasing the friction coefficient known in the art may also be utilized. The inflatable chamber may also be held in position by sutures or clips.

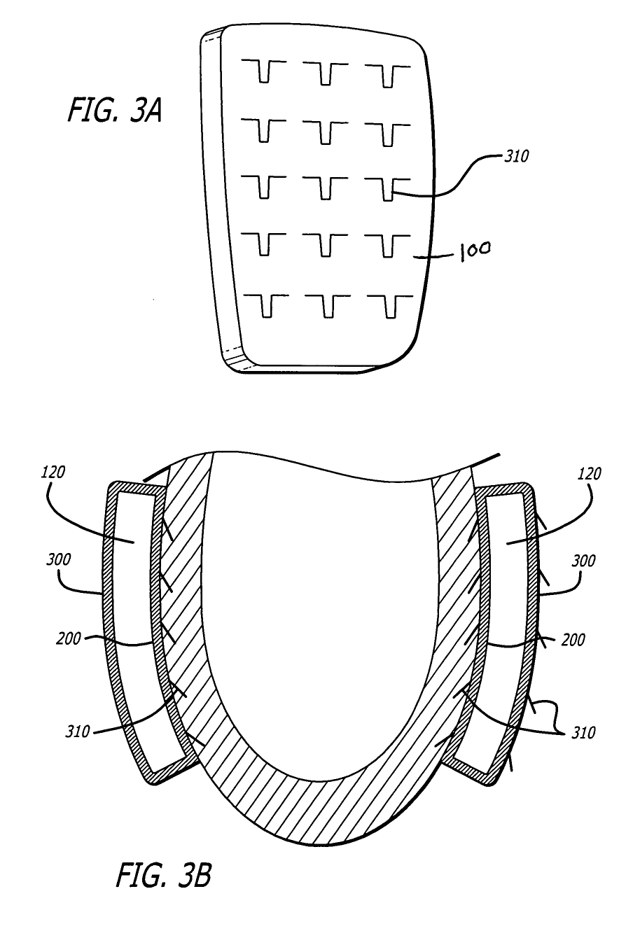

Referring now to FIG. 3A and FIG. 3B, in some embodiments, the exterior of the medial chamber wall 200 or the lateral chamber wall 300 might further include anchoring members 310 to help secure the inflatable chamber 100 in place. Various anchoring members 310 might include for example barbs, pads, or hooks, that will advantageously secure the inflatable chamber 100 in position in the pericardial sac after placement by the clinician. In at least one embodiment, the anchoring members 310 may be intrinsically molded into at least one chamber wall. In yet other embodiments, the anchoring member 310 are connected to the exterior of at least one of the chamber walls by an adhesive, for example.

In one embodiment, the anchoring members 310 may, for example, be similar to the type of barbs described in U.S. Pat. No. 6,595,912 filed Sep. 14, 2001 and entitled “EXPANDABLE CARDIAC HARNESS FOR TREATING CONGESTIVE HEART FAILURE” which is incorporated in its entirety herein by reference. Various non-slip textured surfaces known in the art are suitable anchoring members 310, in yet other embodiments. Biocompatible adhesives may be used to secure the inflatable chamber into position. Textured surfaces encourage the implanted inflatable chamber 100 to remain in place within the pericardial sac, and further encourage the ingrowth of collagen over time. Other methods known in the art may be used to increase the coefficient of friction of the inflatable chamber, thereby keeping the inflatable chamber from slipping out of position on the heart. As adhesions form, the implanted inflatable chamber 100 will become even more securely anchored between pericardium and epicardium.

Referring briefly now to FIGS. 15-16, in one embodiment, there are channels 320 configured between the lateral 300 and medial 200 walls of the inflatable chamber 100 that permit the ingrowth of tissue. The channels may be any shape, for example, round, square, or rectangular. The central lumen of the channels are not in fluid communication with the interior of the inflatable chamber. The channels permit growth of tissue through the channels, however tissue growth into the inflatable chamber is prohibited. The channels also make the walls of the inflatable chamber non-continuous, thereby making it easier to defibrillate a heart that has the inflatable cardiac device 10 implanted.

In yet other embodiments, the inflatable device 10, with or without anchoring members 310, may be secured in place by utilizing an anchoring gel, for example one of the implantable medical adhesives known in the art. One such medical adhesive, by way of example, is a surgical sealant gel available under the brand name CoSeal™. The various anchoring techniques may be applied to the exterior surfaces of either or both the medial chamber wall 200 or the lateral chamber wall 300.

The filling substance in various embodiments can be one of various liquids, gases, or gels currently used in implantable medical devices. Using a liquid, which is less compressible than a gas, will generally result in the inflatable chamber 100 being less compliant and stiffer. Gases are more easily compressed than liquids. A gas filled implant is generally therefore more forgiving and elastic than a liquid filled chamber. However, gases might be readily absorbed from the implant or gas leakage may result in a gas embolism. Generally, filling the inflatable chamber 100 with a gas instead of a liquid will result in a inflatable chamber 100 that is more compliant, more elastic, and less stiff. The filling substance is placed in the cavity 120 of the inflatable chamber 100 before or after implantation in the patient. In one embodiment, the filling substance can be a sponge.

Therefore, completely different volume-pressure relationships within the inflatable chamber 100, and variations in elasticity, compliance, and stiffness of the inflatable cardiac device 10 can be obtained by varying the filling substance used with a particular implanted cardiac device 10. This is advantageous in tailoring the characteristics of the device to a particular patient and to particular medical conditions. In at least one embodiment, the device can be filled with a gel, for example, a silicone gel. In yet other embodiments, saline can be used as the filling substance. Normal saline is considered one of the safest materials to inject or implant into the body. Saline is therefore a preferred liquid filling substance in some embodiments of the invention. Saline implants however sometimes lose volume over time, resulting in an implant becoming soft or rippled. However, saline flows easily and more saline could be added to the inflatable chamber 100 as described in more detail below. In one embodiment, one filling substance may be changed for a different filling substance at a later date. A sponge like material (not shown) or polymer could also be used as the filling substance.

A mildly compressive inflatable chamber 100 is advantageous in providing mild compressive support and conforming to the contours of the heart. Slightly elastic compression of the heart is also advantageous. Therefore, overfilling of the inflatable chamber 100, regardless of the filling substance used, will usually want to be avoided. Overfilling of the inflatable chamber 100 might result in an implant that is too rigid. In yet other embodiments, a filling substance that is at least partially radio-opaque will allow the inflatable chamber 100 to be studied on subsequent radiological studies. This will advantageously allow the physician to inexpensively and accurately dynamically image the outer contours of the heart and the positioning of the inflatable chamber.

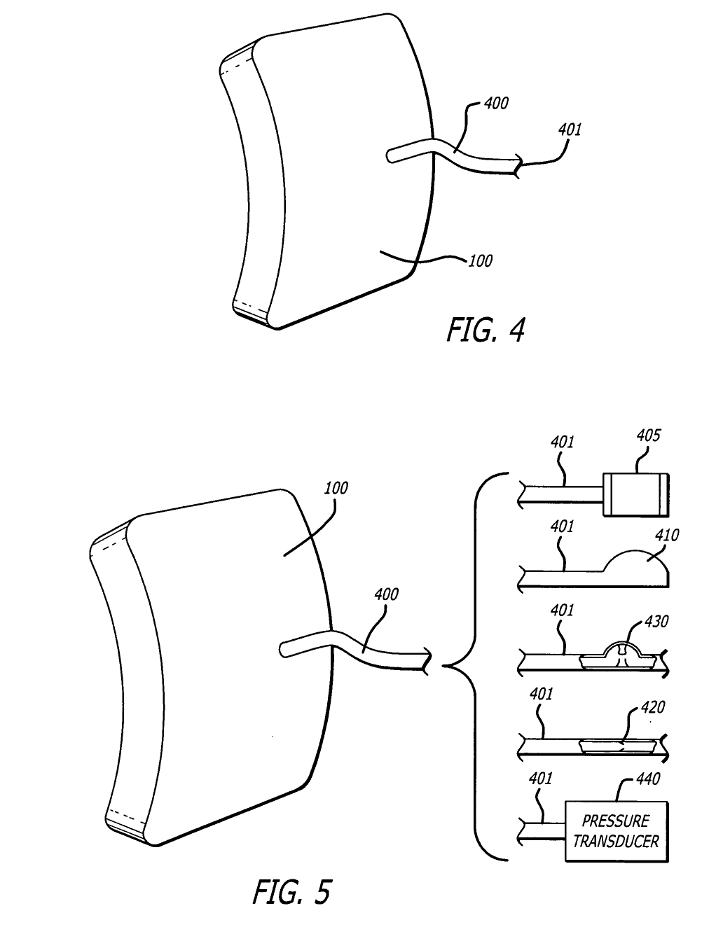

Referring now to FIG. 4, in some embodiments, the inflatable device 10 further includes one or more tubular filling catheters 400. The filling catheter 400 is preferably made from an implantable medical grade material, for example silicone. The filling catheters 400 are elongated tubular member having a generally central inner channel or lumen with an opening on both ends. The channel or lumen extends into and is in fluid communication with the interior of the inflatable chamber 100, whereby fluids may flow to and from the interior of the inflatable chamber 100 through the filling catheters 400. The clinician may fill the inflatable chamber 100 by injecting the filling substance through the filling catheter 400, into the inflatable chamber 100. In some embodiments, the proximal end 401 of the filling catheter 400 may advantageously be brought to the surface during implantation of the inflatable device 10. The inflatable chamber 100 may then be expanded by injection of filling substance without having to pierce the inflatable chamber 100 walls.

The proximal end 401 of the filling catheter 400 may also advantageously be implanted into a subcutaneous location within the patient for subsequent noninvasive access by the clinician. Thereafter, filling substance may be added or removed with a minimally invasive subcutaneous procedure, without having to surgically reopen the pericardium. In some embodiments, the proximal end 401 of the filling catheter 400 may be closed off by the clinician with sutures or surgical clips at the time of implantation of the device, after the inflatable chamber 100 has been satisfactorily inflated.

As illustrated in FIG. 5, in yet another embodiment, the proximal ends of the filling catheters 400 may be connected to an injection port 405, for example, similar to those currently used in medical practice for hypodermic needle access to intravenous lines, made of implantable materials. The injection port 405 may conveniently be located by the clinician by palpation. The injection port may be percutaneously pierced by the clinician utilizing a hypodermic needle, thereby adding or removing filling substances at a time subsequent tom implantation of the inflatable device 10.

In at least one embodiment, the proximal ends of the filling catheters 400 are connected to at least one reservoir 410. Implantable reservoirs are well known in the art. Reservoirs 410 typically are made of medical grade silicone or plastic, with a firm flat base and a domed top. Reservoirs 410 may come in different sizes, holding anywhere from 1 cc. to 100 cc., for example, of fluid. Some available reservoirs are self sealing after puncture by a hypodermic needle. The invention, in some embodiments therefore further includes a reservoir 410 capable of storing additional filling substance. The filling substance in the reservoir 410 is in fluid communication with the lumen of at least one filling catheter 400 and the interior of the inflatable chamber 100. Furthermore, additional filling substance may be percutaneously injected into the reservoir 410, or removed from the subcutaneously implanted reservoir 410. This permits filling substance to be added or removed from the inflatable chamber 100 days, weeks, months, or even years after implantation of the inflatable device 10. A subcutaneous reservoir 410 is also much easier to locate and pierce by a clinician than an injection port 405. A reservoir 410 superficial to the sternum would be very easy for the clinician to palpate due to a paucity of subcutaneous tissue in this location. The reservoir 410 could therefore easily be pierced with a hypodermic needle.

However, repeated percutaneous puncture of a reservoir 410 by hypodermic needles may eventually result in fluid leaking from the reservoir 410 into the subcutaneous space. The reservoirs 410 and subsequently the inflatable chamber 100 could therefore slowly lose volume over time. Therefore, it is advantageous in some embodiments for the invention to further include one or more unidirectional valves 420 interposed between at least one filling catheter 400 and at least one reservoir 410. A unidirectional valve 420 would advantageously allow filling substance to flow in only one direction within a filling catheter 400. The inclusion of an implantable unidirectional valve 420 in the invention permits regulation of the flow of the filling substance within a filling catheter 400. A unidirectional valve 420 permits the filling substance to flow in only one direction, either away from or towards the inflatable chamber 100. The valve 420 could, for example, allow the clinician to fill the inflatable chamber 100, while preventing deflation of the inflatable chamber 100 by reverse flow of filling substance back towards the reservoir 410. Various varieties of valves 420, which may be placed subcutaneously to regulate bodily fluid flow are well known in the art. For example, a wide variety of implantable unidirectional or flow control valves for liquids are commonly utilized for Neurosurgical ventriculo-peritoneal shunts. Examples of these types of valves, which are currently used to regulate intracranial pressure for treatment of hydrocephalus, could be easily modified and used to regulate flows and pressures within the inflatable chamber.

In some embodiments a pressure regulating valve 420 is advantageously placed between the inflatable chamber 100 and a reservoir 410. The pressure regulating valve 420, for example could be used to allow filling substance to flow out of the inflatable chamber 100 to the reservoir 410 if pressures within the inflatable chamber 100 exceeded levels that are dangerous to cardiac function. A pressure regulating valve in the range of 0.1 to 10 mm Hg is usually desirable for a chamber that is placed directly against the epicardium. The pressure regulating valves 420 in some embodiments have a predetermined pressure above which fluid moves out of the inflatable chamber 100 and into at least one reservoir 410. At least one embodiment of the invention including a pressure regulating valve 420 ensures that pressures against the heart are maintained below a preset threshold. If pressure exceeds the threshold, filling substance will move away from the heart towards the reservoir 410. Alternatively, it is also possible to connect at least one pressure regulating valve 420 between the inflatable chamber 100 and reservoir 410 that only allows fluid to move towards the inflatable chamber 100. By placing a higher pressure within the reservoir 410, a pressure regulating valve 420 will direct flow of filling substance towards the inflatable chamber 100 when pressures within the inflatable chamber 100 drop below a predetermined value. It will be understood that by utilizing these pressure regulating valves 420 in various combinations with reservoirs 410, pressures within the inflatable chamber 100 can be maintained within a narrow range of pressures.

Also available in the art are on-off devices 430 that are capable of selectively shutting off flow between a reservoir 410 and a filling catheter 400. On-off devices 430 are frequently combined with a reservoir 410. Typically, flow through the on-off device 430 is shut off by pushing downwards with firm finger pressure on the on-off device. Pressure may be applied on the patients skin that is superficial to the subcutaneously located on-off device 430. For example, the on-off device 430 could be conveniently located subcutaneously just superficial to the sternum where there is little subcutaneous fat and underlying bone for a firm base. The on-off device 430 can be readily palpated in this location by the clinician. When the clinician pushes downward upon the on-off device 430, flow through the on-off device 430 is interrupted. The clinician typically can restore flow through these types of on-off device 430 by pressing downwards on one or more reservoirs 410. The on-off device 430 therefore advantageously offers the clinician a way to interrupt the flow of filling substance between the inflatable chamber 100, and a reservoir 410 or a valve 420.

In some embodiments, one reservoir 410 may be connected with a one way valve 420 that permits only flow of filling substance from the reservoir 410 to the inflatable chamber 100. Another valve 420 permitting only removal of filling substance from the inflatable chamber 100 may be connected with a different reservoir 410. The valves 420 either by themselves or combined with on-off devices 430 allows the clinician to regulate the volume of filling substance within the inflatable chamber 100 without performing an open surgical procedure. For example, with inclusion of a separate reservoir 410, valve 420, and on-off device 430 for filling and removal of fluid from the inflatable chamber 100, it is possible to vary volumes in the inflatable chamber 100 without repeated percutaneous punctures of a reservoir 410 by hypodermic needles. In some embodiments, combinations of on-off devices 430, reservoirs 410, and valves 420 allows a clinician to adjust volumes and pressures in the inflatable chamber 100 without requiring further invasive medical procedures.

In at least one embodiment, the invention includes a pressure transducer 440 capable of sensing pressures within the inflatable chamber 100. The pressure transducer 440 may be connected with a filling catheter 400 or directly connected to the inflatable chamber 100. In yet other embodiments, the clinician will be able to monitor the pressure within the inflatable chamber 100 utilizing an implanted wireless transmitter connected with the pressure transducer 440. Wireless transmitters of this type are known in the art and commonly combined with various implanted medical electronic devices. The transmitter will enable the clinician, with knowledge of the pressure within the chamber, to add or remove fluid in order to customize the pressure in the inflatable chamber to the patient's individual clinical conditions. Furthermore, the pressure transducer 440 provides safety against a clinician over-filling the inflatable chamber 100 and causing deleterious over-compression of the myocardium. For example, the clinician may continuously monitor pressures within the inflatable chamber 100 while percutaneously filling the inflatable chamber 100 through the reservoir 410.

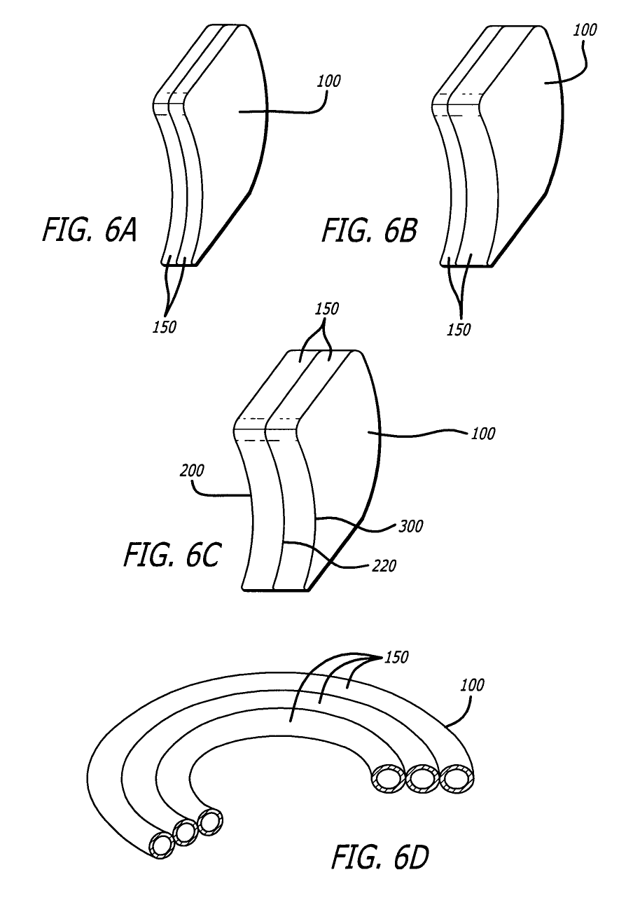

Referring now to FIGS. 6A-6D, the invention in still other embodiments includes an inflatable chamber 100 that has multiple inflatable compartments 150. These multiple inflatable compartments 150 may be provided in a multitude of various shapes and sizes. In one embodiment, the multiple inflatable compartments 150 communicate with each other so that filling substance can flow from one compartment to the next. In another embodiment, the multiple inflatable compartments 150 do not communicate with each other and the contents of a compartment may be isolated from the contents of an adjacent compartment. The inflatable compartments 150 may be connected with each other to form at least one inflatable chamber 100. In some embodiments, the inflatable compartments 150 may be connected together by the manufacturer. In yet other embodiments, the inflatable compartments 150 may be connected together by the clinician, for example with a medical adhesive.

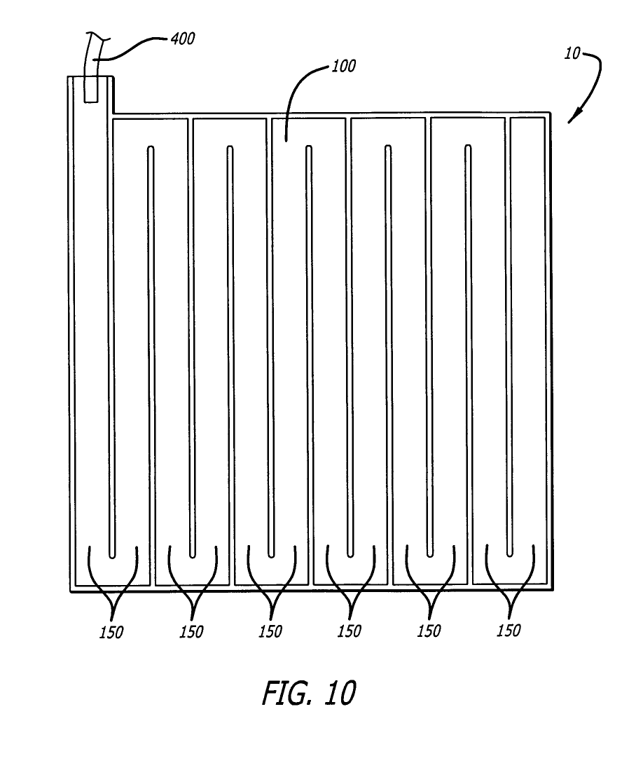

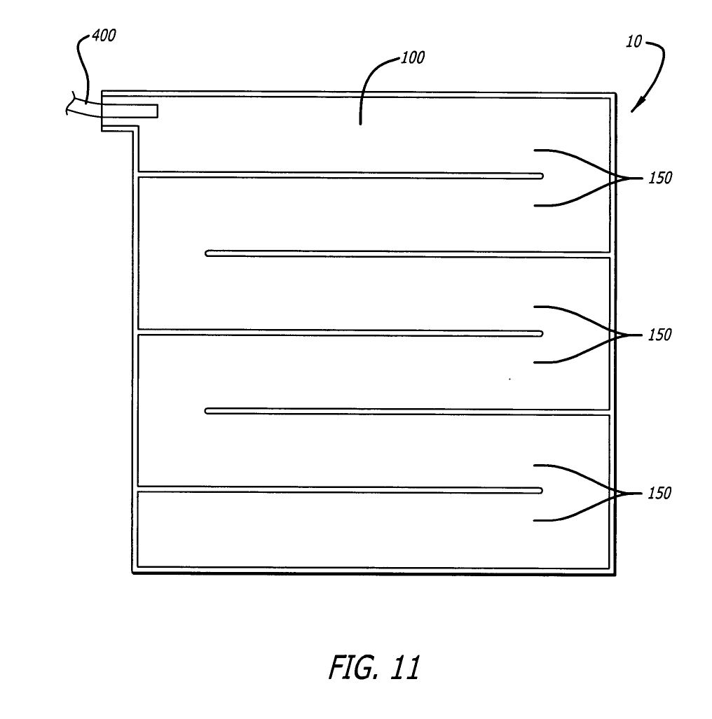

Referring briefly now to FIGS. 10-11, the inflatable compartments may be various widths. In one embodiment the inflatable compartments 150 may be narrow (FIG. 10). For example, in one embodiment, the compartment width may be about six millimeters (0.25 inches). In one embodiment, the inflatable compartment may be wide (FIG. 11). For example, in one embodiment the compartment width may be about twelve millimeters (0.50 inches). Many other compartment widths are possible and the above stated widths are not meant to be limiting. In yet another embodiment (not shown), an inflatable device may include combinations of wide and narrow compartments.

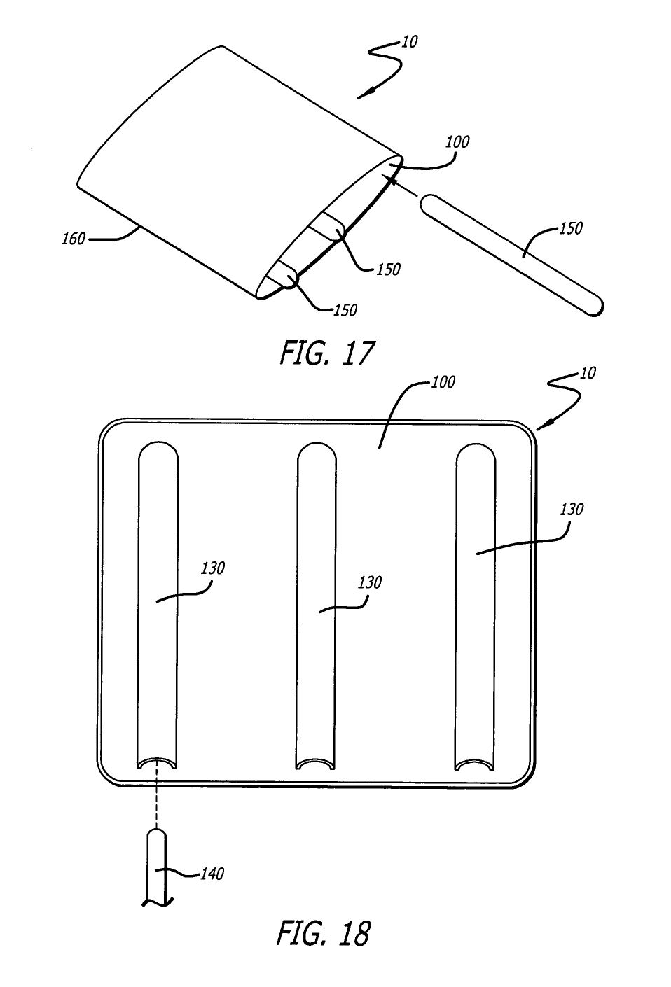

Referring now to FIG. 17, in one embodiment, the inflatable compartments 150 may be individually inserted into a pouch 160. In one embodiment, a plurality of inflatable compartments are inserted into the pouch. In one embodiment, the inflatable chambers may lay sided by side in the pouch 160 without connecting the inflatable chambers 150 together. In one embodiment, the inflatable chambers may be connected together before or after insertion into the pouch, for example, with an adhesive. The inflatable compartments 150 may be, by way of example, tubular shaped or they may be any number of other shapes. The pouch 160 may be placed in the pericardial sac and the inflatable compartments 150 then inserted one by one into the pouch 160. In another embodiment, the inflatable compartments 150 are loaded into the pouch 160 before the pouch is implanted into the pericardial sac. The pouch 160 may be sealed with sutures, clips, adhesive, Velcro™, or a Zip-lock™ type closure similar to the kind used in food storage bags.

The multiple inflatable compartments 150 may be columnar, round, rectangular prisms, or other advantageous shapes. For example, the inflatable chamber 100 may in some embodiments include rows of multiple columnar shaped inflatable compartments 150 connected side by side at the long edge. As shown in FIG. 6D, in some embodiments concentric ring shaped inflatable compartments 150 are connected together to form a substantially circular inflatable chamber 100. Connecting together various shape and size compartments 150 permits great variability in designing an inflatable cardiac device that is specific for the patient's needs.

It is also possible to designate one or more compartments 150 within the inflatable chamber 100 as pressure sensing compartments 150. The pressure transducer 440 could be connected directly to the compartment 150, or connected to the compartment 150 by a catheter. Knowledge of pressures in the compartments 150 and inflatable chamber 100 is advantageous in alerting the clinician to add or remove filling substance, or even to change one filling substance for another. Different compartments may also be filled with different kinds of filling substance, thereby providing great control and variety over compliance, rigidity, and pressures.

As illustrated in FIG. 6C, in yet other embodiments, two or more compartments sharing a common wall 220 may be provided. In one embodiment, rectangular prism shaped compartments 150, less thick than long and wide in dimensions are connected together. In some embodiments, these mattress shaped inflatable compartments 150 are combined wherein the medial chamber wall 200 of the inflatable chamber 100 is substantially comprised of the wall of one compartment 150, the lateral chamber wall 300 of the inflatable chamber 100 is substantially comprised of the wall of another compartment 150, and there is a shared common wall 220 between the two inflatable compartments 150.

The advantage of the inflatable chamber 100 comprising multiple inflatable compartments 150 is that one side or certain areas of the inflatable chamber 100 can be made more stiff or more compliant by varying the volumes, pressures, and type of filling substance within various the various inflatable compartments 150. Volume may be added or removed from individual inflatable compartments 150 to shape and adapt the inflatable chamber 100 to the needs of the patient. Different compartments 150 may also be filled with different filling substances in order to provide different volume-pressure relationships in different compartments 150. For example, one compartment 150 may be filled with a liquid to make that compartment 150 less compliant, and another compartment 150 filled with a gas to make the other compartment 150 more elastic. Furthermore, by varying the thickness of at least one compartment wall, some compartments 150 may be made more pliable than others, again varying the volume-pressure relationships from compartment 150 to compartment 150. Finally, in some embodiments, one or more compartments 150 may have a semi-permeable wall, wherein drugs contained in the compartment 150 are capable of diffusion into the pericardial space.

In another embodiment, at least one compartment 150 or the supplemental layer 350 of the invention may also be used for localized delivery of various drugs and other biologically active agents. The drugs used may be, for example, useful for the treatment of congestive heart failure or other heart treatment. Drugs, biopharmaceuticals, and any other physiological process modifying agents may be delivered from a biomaterial composition to a local tissue or into systemic circulation after absorption by local tissues. The biomaterial delivered drug, biopharmaceuticals, therapeutic agents or physiological process modifying agents can be anti-infective, anti-inflammatory, anti-proliferative, anti-angiogenic, anti-neoplastic, anti-scaring, scar-inducing, tissue-regenerative, anesthetic, analgesic, immuno-modulating agents and neuro-modulating. Further examples of drugs include those described in U.S. Pat. No. 6,759,431, and U.S. Published Application 2004/0219214 which are incorporated by reference herein.

In one embodiment, the biomaterial delivered into the intrapericardial space by the inflatable device comprises a drug for the treatment of congestive heart failure. Examples of drugs used for congestive heart treatment include Furosemide, Hydrochlorothiazide, Metolazone, Digoxin, Dopamine, Dobutamine, Inamrinone, Milrinone, Captopril, Enalapril, Lisinopril, Nitroprusside, Alprostadil, DITPA, or 3, 5-diiodothyropropionic acid.

In yet another embodiment, a biomaterial delivered into the intrapericardial space by the inflatable device comprises a fibrosis-inducing agent causing the formation of fibrotic capsule tissue on the surface of the myocardium and in the intrapericardial space. Examples of fibrosis-inducing reagents include chitosan, fibronectin, bleomycin, polylysine, silk protein, talk powder. Other examples of fibrosis-inducing agents include those described in U.S. Published Applications 2005/0169958 and 2005/0169959, which are incorporated by reference herein.

In yet other embodiments, endogenous factors and drugs that might affect myocardial collagen turnover may be delivered by the inflatable device. These include endogenous factors drugs, and collagen synthesis inhibitors, for example, bradykinin, adenosine, P4H inhibitors, nitric oxide, catecholamines, MMP inhibitors, Interferon, ACE inhibitors, parathormone, ARBs, Thyroid hormone, bradykininase inhibitors, glucocoricoids, endothelin antagonists, steroid hormones, chymase inhibitors, or vasopeptidase inhibitors. These may further include collagen breakdown promoters, for example, MMPs, TIMP inhibitors, bradykinin, or Adriamycin. These may also include collagen synthesis promoters, for example, P4H, growth hormone, TGF, bradykinin inhibitor, angiotensin II, aldosterone, ACE, chymase, endothelin-1, CTGF, PDGF, EGF (epidermal growth factor), TGF- (transforming growth factor-), bFGF (basic fibroblast growth factor), IGF (insulin-like growth factor), or ascorbic acid. These may further include collagen breakdown inhibitors, for example, TIMPs, Recombinant TIMP, phenytonin, retinoids, or MMP inhibitors.

The inflatable compartments 150 in some embodiments may communicate with each other such that liquid or gas in one compartment 150 will freely flow into another compartment 150, similar to the design of an air mattress. In other embodiments, an inflatable compartment 150 is not in fluid communication with another inflatable compartment 150, such that each compartment 150 may be filled or emptied separately from the other. The size and shape of the inflatable chamber 100 may therefore be regulated to a degree by varying the type of filling substance and the injection volume of each inflatable compartment 150. Furthermore, the thickness of the inflatable chamber 100 may also be regulated by varying not only the size of each compartment 150, but also by varying the injection volume of each individual inflatable compartment 150. In one embodiment the inflatable chamber mattress structure is made out of thermoplastic polyurethane that is heat sealed.

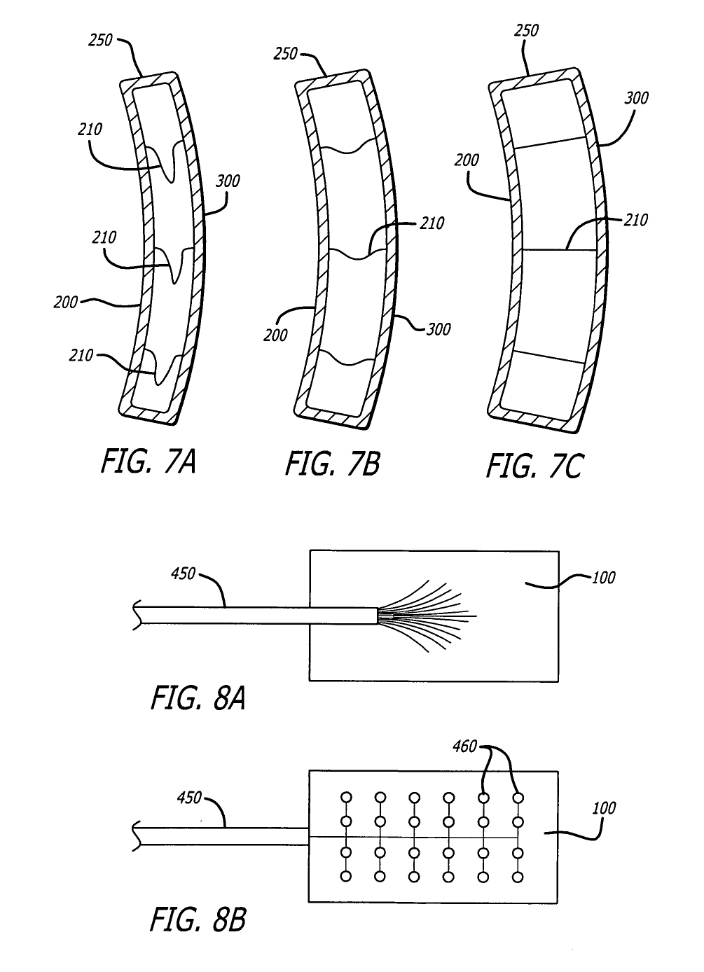

As illustrated in FIGS. 7A-7C, in at least one embodiment, interconnecting members 210 are provided that limit the expansion of the medial wall 200 away from the lateral wall 300. The thickness of the inflatable chamber 100, from medial chamber wall 200 to lateral chamber wall 300 can be varied and controlled. The interconnecting members 210 are bands running between at least two walls which mechanically limit the separation of the walls to the length of the interconnecting member 210. The interconnecting members 210 prevent the medial chamber wall 200 from separating too far apart from the lateral chamber wall 300. Interconnecting members 210 can also be placed within compartments 150. The advantage of the interconnecting members 210 is that the inflatable chamber 100 or compartment 150 cannot be over inflated beyond a predetermined thickness. The interconnecting members 210 further provide a safety mechanism preventing excessive compression of the myocardium by the inflatable cardiac device.

In yet other embodiments, by altering the number, size, shape, and arrangement of inflatable compartments 150, and further varying the inclusion of interconnecting members 210, the rigidity or flexibility of the inflatable chamber 100 may be controlled. Generally, the interconnecting members 210 will restrict the distension of the inflatable chamber 100. Therefore, as more volume is added to the inflatable chamber 100, the inflatable chamber 100 will tend to stiffen rather than distend. After implantation, the inflatable chamber 100 could be made more or less elastic by varying the volume and type of filling substance. This is advantageous in reshaping the heart without applying undesirable pressures to the myocardium.

Referring now to FIG. 8A-8B, yet another embodiment of the invention is the inclusion of a drug delivery catheter 450 connected with the inflatable chamber 100. In one embodiment, the drug delivery catheter 450 may be in fluid communication with the inside of the inflatable chamber 100. At least the medial chamber wall 200 or the lateral chamber wall 300 may include a semi-permeable membrane that allows the drug to diffuse out of the inflatable chamber 100 over time. In yet other embodiments, the drug delivery catheter 450 may be connected to the outside of the inflatable chamber 100. The drug delivery catheter 450 could either carry a drug to the pericardial space or carry an anchoring gel to secure the inflatable chamber 100 in position. In at least one embodiment, the invention may further include a baffle system 460 (FIG. 8B) for evenly distributing a drug from the drug delivery catheter 450 onto the surface of the inflatable chamber 100. Furthermore, the proximal end of the drug delivery catheter 450 may be connected with one or more subcutaneous reservoirs 410 that contain a supply of drugs. Referring specifically to FIG. 8A, the ends of the drug delivery catheter 450 which are connected to the outside of the inflatable chamber 100 may, in some embodiments, empty into the pericardial sac. Drugs can therefore advantageously be delivered directly to the myocardium or pericardium after implantation of the inflatable device 10. In yet other embodiments, at least one drug delivery catheter 450 may be further connected with an active drug pump (not shown) of the type currently known in the art. These drug pumps are typically implanted subcutaneously, and are capable of being programmed and refilled with a variety of drugs.

Diseased hearts often have several maladies. One malady that is not uncommon is irregularity in heartbeat caused by irregularities in the electrical stimulation system of the heart. For example, damage from a cardiac infarction can interrupt the electrical signal of the heart. In some instances, implantable devices, such as pacemakers, help to regulate cardiac rhythm and stimulate heart pumping. A problem with the heart's electrical system can sometimes cause the heart to fibrillate. During fibrillation, the heart does not beat normally, and sometimes does not pump adequately. A cardiac defibrillator can be used to restore the heart to normal beating. An external defibrillator typically includes a pair of electrode paddles applied to the patient's chest. The defibrillator generates an electric field between electrodes. An electric current passes through the patient's heart and stimulates the heart's electrical system to help restore the heart to regular pumping.

In some patients that are especially vulnerable to fibrillation, an implantable heart defibrillation device may be used. Typically, an implantable heart defibrillation device includes an implantable cardioverter defibrillator (ICD) or a cardiac resynchronization therapy device (CRT-D) which usually has only one electrode positioned in the right ventricle, and the return electrode is the defibrillator housing itself, typically implanted in the pectoral region. Alternatively, an implantable defibrillation device includes two or more electrodes mounted directly on, in or adjacent the heart wall. If the patient's heart begins fibrillating, these electrodes will generate an electric field therebetween, creating a shock to the heart.

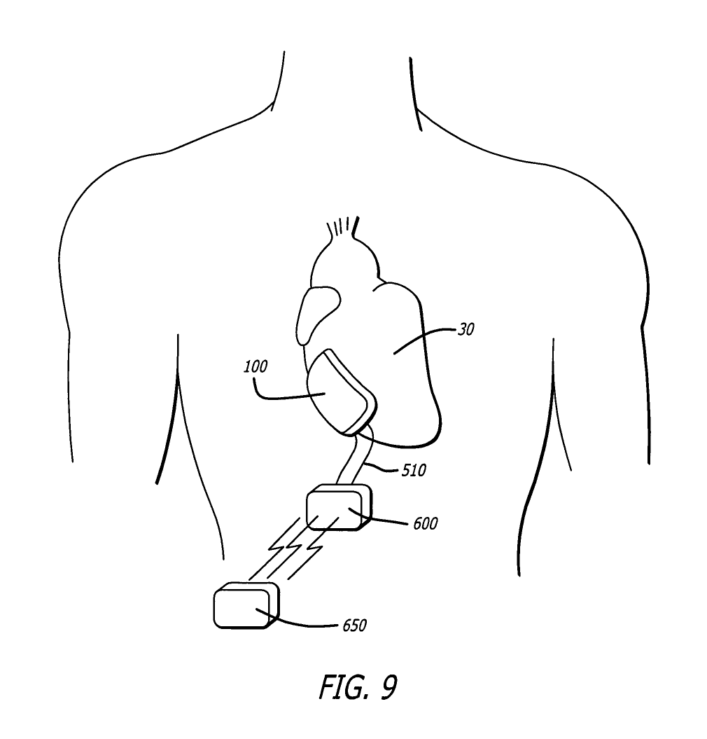

Therefore, referring now to FIG. 9, still other embodiments of the invention include an implantable electronic medical device 600. The implantable electronic medical device 600 may be connected by electrical leads 500 with at least one electrode 510 positioned on at least one surface of the inflatable chamber 100. For example, in at least one embodiment the present invention includes a cardiac rhythm management device for treating any number of irregularities in heart beat due to, among other reasons, congestive heart failure. Thus, the cardiac rhythm management device, connected with the inflatable device 10, can include one or more of the following: an implantable cardioverter/defibrillator; a cardiac pacemaker used for sensing cardiac function and providing pacing stimuli to the heart; and a combined implantable cardioverter/defibrillator and pacemaker, to provide a defibrillation shock and/or pacing/sensing functions. The electronic electrodes 510 and leads 500 are also advantageous in measuring the impedance across the heart, which may give an early indication of heart failure.

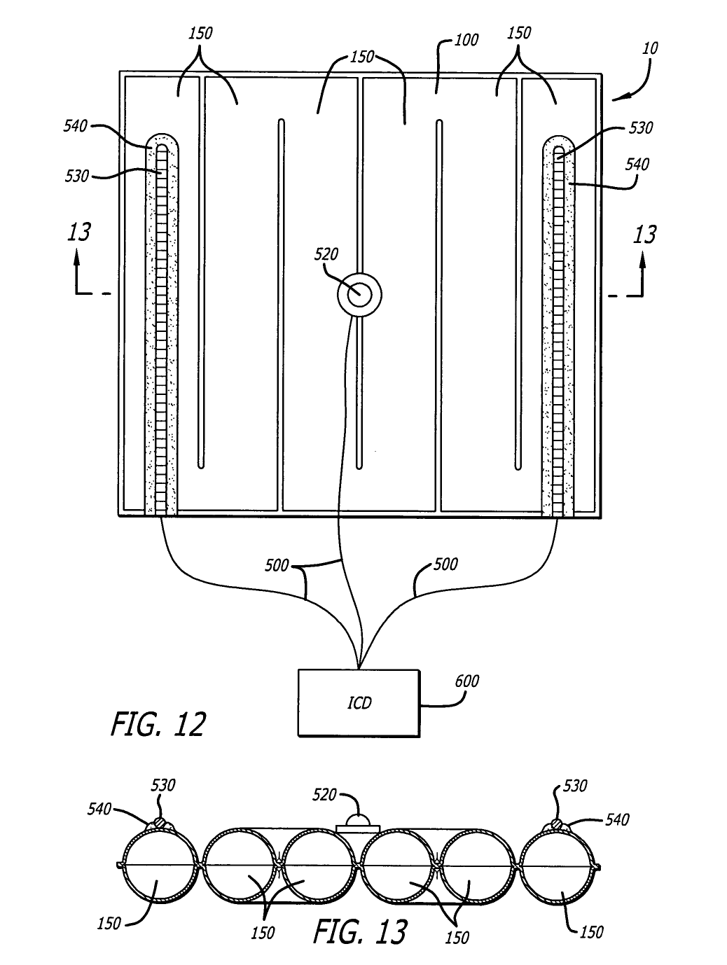

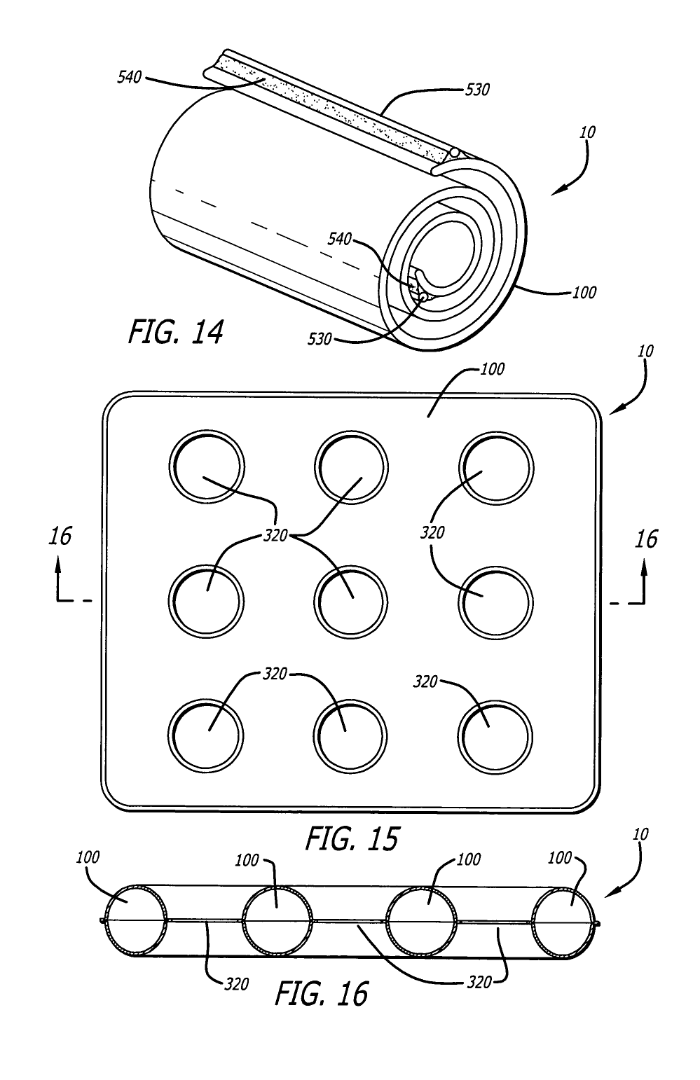

Referring now to FIGS. 12-14, in one embodiment, the inflatable device 10 includes at least one sensing electrode 520 and at least one defibrillating electrodes 530. The defibrillating electrodes may include elongated electrically conducting members that are attached to the epicardial side or lateral wall 300 of the inflatable chamber. The defibrillating electrodes 530 may include reinforced sheaths 540 that help attach the electrically conducting members to the inflatable chamber 100. The inflatable device including the electrodes 520, 530 may be longitudinally rolled up or folded in a collapsed configuration and delivered to the heart with minimally invasive or percutaneous techniques. The inflatable device having electrodes may be inflated after delivery onto the heart. One or more sensing electrode 520 is configured to sense the electrical activity of the heart. One and preferably two defibrillating electrodes 530 is configured to provide an electrical impulse to the heart, as well known in the art. The electrodes are preferably in electrical communication with an implantable electronic medical device 600 through leads 500.

At least one embodiment of the inflatable device 10 would therefore advantageously further include one or more electrodes 510, 520, 530 on the medial surface of the inflatable chamber 100 and at least one lead 500 connecting the leads to an implantable electronic medical device 600, including a defibrillator that is positioned subcutaneously. The electrodes would sense electrical cardiac activity and transmit the information to the implantable electronic medical device 600 defibrillator. The defibrillator would generate an electrical shock back to the heart when necessary to maintain a safe cardiac rhythm. The leads 500 on the inflatable device 10 could also be electrically connected with an implantable electronic medical device 600 having a transmitter. The transmitter may be in electrical communication with an external receiver 650 that is worn on the exterior of the patient. The external receiver 650 may sense the heart's electrical activity transmitted from the implanted transmitter and sound an alarm that will notify the patient to call for medical attention if one or more potentially dangerous rhythms, for example atrial fibrillation, are detected. Furthermore, the external receiver 650 may also advantageously include a telephone communication device that automatically sends a notification to a relative, a physician, or a monitoring station. An emergency signal and GPS location could be sent by the receiver requesting urgent help from paramedics if a life threatening cardiac rhythm is detected. The notification will allow early intervention when there is evidence of a dangerous cardiac rhythm taking place in the patient. The proximal end of the leads 500 could also be connected to a pacemaker in the implantable electronic medical device 600. In at least one embodiment, the external receiver 650 may also include a transmitter that sends information to the implantable electronic medical device 600, which has a receiver. The implantable electronic medical device 600 can then be programmed or controlled from external to the patient's skin.

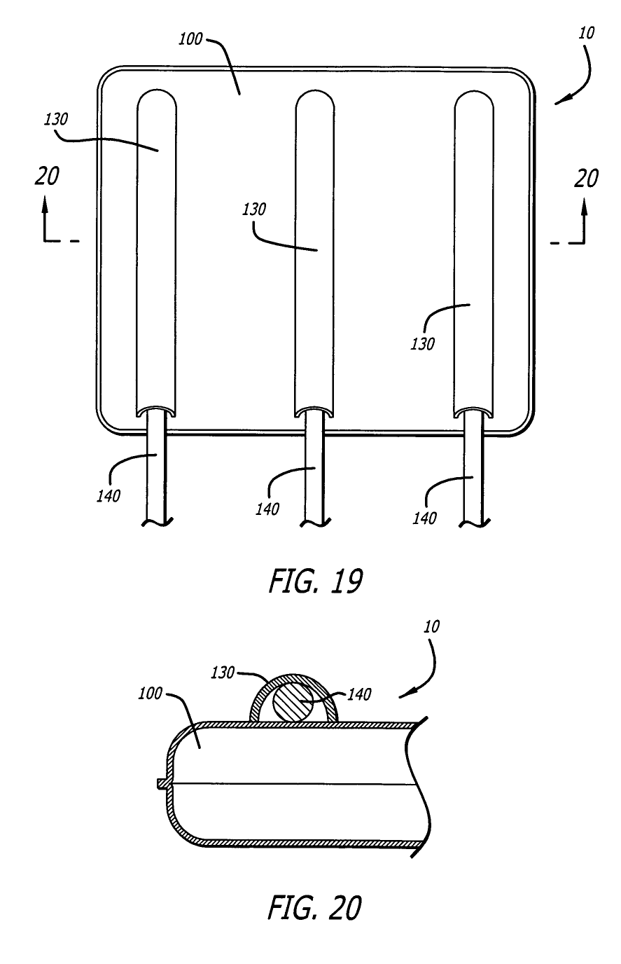

Referring briefly now to FIGS. 18-20, in one embodiment, the inflatable cardiac device 10 includes longitudinal sheaths 130 for receiving at least one delivery rod 140. The delivery rod is elongated and in one preferred embodiment is tubular and may be rounded on at least one end. One or more delivery rod may be inserted into the longitudinal sheath before or during implantation of the inflatable device. The delivery rods may be used by the surgeon to manipulate the inflatable chamber into proper position within the pericardium. The delivery rods may then be removed from within the longitudinal sheaths.

Another aspect of the invention is a method of treating a diseased heart with minimally invasive surgery. The method of treating the diseased heart comprises providing an inflatable cardiac device 10 having at least one inflatable chamber 100 and inserting the inflatable cardiac device into a patient. The method further includes positioning the inflatable cardiac device in a collapsed configuration adjacent a portion of a patient's heart and inflating the inflatable chamber with a filling substance. In at least one embodiment, the method further includes leaving the inflatable cardiac device implanted within the patient. The inflatable cardiac device may be implanted within the patient for days, weeks, months, or years.

In at least one embodiment, the method includes minimally invasive delivery of the inflatable cardiac device 10 through a canula 20. The canula is passed through a small incision in the patients skin into the pericardial sac using minimally invasive surgical techniques. The inflatable cardiac device may be rolled up about an axis and passed through the lumen of the canula into the pericardial space. The inflatable cardiac device is then unrolled and inflated with filling substance. In one embodiment the inflatable cardiac device is delivered percutaneously. In one embodiment, the inflatable cardiac device may be delivered via a subxiphoid approach. In yet one further embodiment, the inflatable cardiac device may be folded up for delivery. In one additional embodiment, the inflatable cardiac device may be positioned on the heart in a standard open chest procedure, for example during thoracotomy or during open heart surgery.

In one embodiment, the method further includes using an endoscope to aid in accurate positioning of the inflatable cardiac device 10. In at least one embodiment, the inflatable cardiac device is positioned upon the epicardium 30 using non-invasive imaging, for example echocardiography and/or fluoroscopy. In one embodiment, echocardiography is used to aid in visualizing and guiding the implantation of the device. In yet another embodiment, the implant is at least partially radiopaque and the method includes using fluoroscopy to aid in visualizing and guiding the implantation of the device. In at least one embodiment, the method includes implanting an inflatable cardiac device including filling catheters 400 and reservoirs 410. The reservoirs may be implanted subcutaneously for easy access by the clinician. In yet other embodiments, the method further includes implanting valves 420, on-off devices 430, and/or pressure transducers 440.

In one further embodiment, the method includes providing a inflatable device 10 including a pouch 160. The pouch is positioned between the epicardium 30 and the pericardium 40. The pouch may then be filled with a plurality of inflatable compartments 150. The inflatable compartments 150 may be connected with each other, for example with an adhesive, or inserted into the pouch in an unconnected configuration. The method may further include sealing the pouch 160 with, for example, adhesive, sutures, surgical clips, or a zip-lock closure (not shown) configured on the pouch.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

What is claimed:

1. An inflatable cardiac device configured for minimally invasive delivery and long term implantation upon the heart, comprising:
an implantable inflatable chamber including a medial chamber wall connected to a lateral chamber wall by at least one peripheral wall.

2. The inflatable cardiac device of claim 1, wherein the inflatable chamber has a first deflated configuration and a second inflated configuration, wherein the inflatable chamber can be inserted into the body through a canula.

3. The inflatable cardiac device of claim 1, wherein one of the chamber walls is more rigid the other chamber wall.

4. The inflatable cardiac device of claim 1, wherein an exterior surface of at least one wall is configured having an increased coefficient of friction.

5. The inflatable cardiac device of claim 1, further including:
at least one electrode disposed on the medial chamber wall; and
an electrical lead having two ends, wherein one end of the electrical lead is connected to the electrode, and the other end of the electrical lead is capable of connection with an implantable electronic medical device.

6. The inflatable cardiac device of claim 1, further including a supplemental layer connected with at least one of the chamber walls.

7. The inflatable cardiac device of claim 6, wherein the supplemental layer includes anchoring members.

8. The inflatable cardiac device of claim 6, wherein the supplemental layer includes a pharmacologic agent.

9. An inflatable cardiac device, comprising:
an implantable inflatable chamber including a medial chamber wall connected with a lateral chamber wall; and
a plurality of anchoring members disposed upon the exterior of at least one of the chamber walls.

10. The inflatable cardiac device of claim 9, further including:
at least one electrode disposed on at least one of the chamber walls; and
an electrical lead having two ends, wherein one end of the electrical lead is connected to the electrode, and the other end of the electrical lead is capable of connection with an implantable electronic medical device.

11. The inflatable cardiac device of claim 9, further including at least one filling catheter having a generally central axial lumen connected with the inflatable chamber, wherein the lumen of the filling catheter is in fluid communication with the interior of the inflatable chamber.

12. The inflatable cardiac device of claim 11, further including a pressure transducer connected with the filling catheter.

13. The inflatable cardiac device of claim 11, further including at least one implantable reservoir connected with the filling catheter, wherein the lumen of the filling catheter is in fluid communication with the interior of the reservoir.

14. The inflatable cardiac device of claim 13, further including at least one valve connected between the filling catheter and the reservoir.

15. The inflatable cardiac device of claim 13, further including at least one on-off device connected between the filling catheter and the reservoir.

16. A system for minimally invasive surgical insertion of an inflatable cardiac device, comprising:
a canula having an axial lumen sized to pass a deflated implantable inflatable cardiac device therethrough;
the inflatable cardiac device, including an inflatable chamber configured for long term implantation upon a patient's heart, the inflatable chamber having a medial chamber wall connected with a lateral chamber wall; and
at least one catheter connected with the inflatable chamber.

17. The system for minimally invasive surgical insertion of an inflatable cardiac device of claim 16, wherein the catheter is a drug delivery catheter, and wherein the catheter is further connected with at least one implantable reservoir.

18. The system for minimally invasive surgical insertion of an inflatable cardiac device of claim 16, wherein the catheter is a drug delivery catheter, and the wherein the catheter is further configured for connection with an implantable drug pump.

19. The system for minimally invasive surgical insertion of an inflatable cardiac device of claim 16, further including an anchoring gel.

20. An inflatable cardiac device, comprising:
a pouch, the exterior of the pouch including a surface having an increased coefficient of friction; and
at least two inflatable compartments configured to be positioned side by side inside the pouch.

21. The inflatable cardiac device of claim 20, further including:
at least one electrode connected with at least one surface of the pouch; and
an electrical lead having two ends, wherein one end of the electrical lead is connected to the electrode, and the other end of the electrical lead is capable of connection with an implantable electronic medical device.

22. The inflatable cardiac device of claim 20, further including a pressure transducer connected with at least one compartment.

23. The inflatable cardiac device of claim 20, further including at least one filling catheter, having a generally central axial lumen, connected with at least one of the inflatable compartments, the lumen of the filling catheter being in fluid communication with the interior of the inflatable compartment.

24. An inflatable cardiac device configured for minimally invasive surgical insertion, comprising:
an implantable inflatable chamber including a medial chamber wall and a lateral chamber wall, at least one of the walls including an exterior surface having an increased coefficient of friction; and
at least two inflatable compartments connected to each other by at least one common wall.

25. The inflatable cardiac device of claim 24, further including:
at least one electrode connected with the exterior of the medial chamber wall; and
an electrical lead, wherein one end of the electrical lead is connected to the electrode, and an opposite end of the lead is capable of connection with an implantable electronic medical device.

26. The inflatable cardiac device of claim 24, further including at least one pressure transducer capable of measuring a pressure within at least one compartment.

27. The inflatable cardiac device of claim 24, further including at least one filling catheter having a generally central axial lumen, connected with at least one of the inflatable compartments.

28. The inflatable cardiac device of claim 27 further including at least one implantable reservoir connected with the filling catheter.

29. The inflatable cardiac device of claim 28 further including a valve connected between the filling catheter and the reservoir.

30. The inflatable cardiac device of claim 28 further including at least one on-off device connected between the filling catheter and at least one reservoir.

31. The inflatable cardiac device of claim 24, further including a drug delivery catheter connected with at least one inflatable compartment.

32. The inflatable cardiac device of claim 31, further including at least one implantable reservoir connected with the drug delivery catheter.

33. The inflatable cardiac device of claim 31, further including an implantable drug pump connected with the drug delivery catheter.

34. A method of treating a diseased heart, comprising:
providing an inflatable cardiac device having at least one inflatable compartment;
inserting the inflatable cardiac device into a patient;
positioning the inflatable cardiac device in a collapsed configuration between the patient's epicardium and pericardium;
inflating the inflatable chamber with a filling substance; and
leaving the inflatable cardiac device implanted within the patient.

35. The method of claim 34, wherein the inflatable cardiac device is inserted into the patient through a canula.

36. The method of claim 34, wherein the inflatable cardiac device is inserted into the patient minimally invasively.

37. The method of claim 34, wherein the inflatable cardiac device is inserted percutaneously.

38. The method of claim 34, further including connecting a filling catheter to at least one reservoir and implanting the filling catheter and the reservoir within the patient.

39. The method of claim 34, further including delivering a drug to the patient using the inflatable cardiac device.

40. The method of claim 34, wherein the inflatable cardiac device is positioned with the assistance of non-invasive imaging.

41. The method of claim 34, further including connecting at least one sensing electrode and at least one defibrillating electrode to an implantable electronic medical device.

42. The method of claim 34, further including measuring the impedance of the heart through at least one of the electrodes.

43. The method of claim 34, further including deflating the inflatable cardiac device after implantation within the patient.

44. The method of claim 34, further including inserting at least one inflatable compartment into an inflatable cardiac device including a pouch.

45. The method of claim 34, wherein the inflatable cardiac device is positioned using rods inserted into sheaths.

46. The method of claim 34, wherein the inflatable cardiac device is implanted in the patient for at least one week.
